(19) United States
(12) Patent Application Publication
(10) Pub. No.: US 2022/0151888 A1
(43) Pub. Date: May 19, 2022

(54) SUGAR-FREE LIP SCRUB COMPOSITIONS AND USES THEREOF

(71) Applicant: Tommy Marquez, Las Vegas, NV (US)

(72) Inventor: Tommy Marquez, Las Vegas, NV (US)

(21) Appl. No.: 17/526,266

(22) Filed: Nov. 15, 2021

Related U.S. Application Data
(60) Provisional application No. 63/114,456, filed on Nov. 16, 2020.

(51) Int. Cl. A61K 8/34; A61Q 19/00

(52) U.S. Cl. CPC A61K 8/345; A61K 2800/87; A61Q 19/001

(57) ABSTRACT

Disclosed herein are lip scrub compositions comprising an exfoliating agent that is not a granulated sugar. The lip scrub compositions disclosed herein may further comprise one or more ingredients selected from an emollient, flavoring agent, colorant, abrasive, drug agent, thickening agent, and vitamin.

### CROSS-REFERENCE TO RELATED APPLICATION

This application claims priority under 35 U.S.C. § 119(e) to U.S. Provisional Patent Application No. 63/114,456, filed Nov. 16, 2020, the entire contents of which are incorporated herein by reference.

### FIELD OF THE INVENTION

Described sugar-free lip scrub compositions and uses thereof.

### BACKGROUND OF THE INVENTION

The following discussion is merely provided to aid the reader in understanding the disclosure and is not admitted to describe or constitute prior art thereto.

There are a wide variety of lip care products on the market, including lip scrubs. Most lip scrubs are sugar-based and comprise sugar granules. Lip scrub compositions may be contained in jars and are topically applied using fingers or spoon-like applicators.

Common lip scrubs typically comprise no less than 70% w/w of granular sugar, with the granular sugar being used to exfoliate dead skin from lips. Lip scrub compositions containing less than 70% w/w/ of granular sugar, such as lip scrubs comprising mostly emollients, are often unable to exfoliate dead skin off lips.

A major drawback of common lip scrubs comprising granular sugar is that dissolution or melting of the sugar component creates an acid that can damage tooth enamel. Oral bacteria can break down sugar-containing foods to produce acid in as little as 20 seconds. Over time, the continued production of acid from the reaction of sugary products, such as lip scrubs, and oral bacteria results in tooth decay, which may lead to increased cavities and gum disease. As it is almost impossible to rub apply a sugar-based lip scrub to the lips without any of the lip scrub entering the mouth and contacting the teeth, there is a need to develop a sugar-free lip scrub that allows for exfoliation of the lips without increasing cavities and gum disease. The present invention provides such sugar-free lip scrub compositions.

### SUMMARY OF THE INVENTION

Disclosed herein is a lip scrub composition comprising an exfoliating agent that is a granulated sugar alcohol, wherein the lip scrub composition comprises less than about 95%, 90%, 80%, 70%, 60%, 50%, 40%, 30%, 20%, or 10% w/w sugar.

In some embodiments, the sugar alcohol is selected from erythritol, xylitol, mannitol, sorbitol, lactitol, isomalt, maltitol and hydrogenated starch hydrolysates (HSH). In some embodiments, lip scrub composition comprises at least about 40%, 50%, 55%, 60%, 65%, 70%, 75%, 80%, 85%, 90%, or 95% w/w of the exfoliating agent. In some embodiments, the lip scrub composition comprises about 75% to about 85% w/w of the exfoliating agent. In some embodiments, the exfoliating agent is erythritol. In some embodiments, the lip scrub composition comprises at least about 70%, 75%, 80%, 81%, 82%, 83%, 84%, or 85% w/w of erythritol. In some embodiments, the lip scrub composition comprises at least about 81% w/w of erythritol. In some embodiments, the lip scrub composition comprises between about 70% to about 85%, about 75 to about 85%, or about 80% to about 85% w/w of erythritol.

In some embodiments, the lip scrub composition comprises less than about 90%, 80%, 70%, 60%, 50%, 40%, 30%, 20%, or 10% w/w Citrus limon peel powder.

In some embodiments, the lip scrub composition comprises less than about 90%, 80%, 70%, 60%, 50%, 40%, 30%, 20%, or 10% w/w Oryza sativa rice powder.

In some embodiments, the lip scrub composition comprises less than about 90%, 80%, 70%, 60%, 50%, 40%, 30%, 20%, or 10% w/w allulose.

In some embodiments, the lip scrub composition is edible.

In some embodiments, the lip scrub composition is safe for human consumption.

In some embodiments, the lip scrub composition further comprises an emollient, flavoring agent, thickening agent, essential oil, colorant, vitamin, humectant, preservative, or any combination thereof.

In some embodiments, the lip scrub composition comprises an emollient. In some embodiments, the emollient is selected from: Aloe vera oil, aprikot kernel oil, argan oil, avocado oil, babassu oil, blueberry seed oil, broccoli seed oil, Butyrospermum parkii unsaponifiables, Camellia kissi seed oil, Camelina oil, canola oil, carrot seed oil, castor oil, celery seed oil, coconut oil, Cocos nucifera (Coconut) Oil, cranberry seed oil, cucumber seed oil, dimethicone, flaxseed oil, glycerin, grapeseed oil, green tea oil, hazelnut oil, Helianthus annuus seed oil, hemp oil, hydrogenated moringa oil esters, jojoba oil, lanolin, lavender oil, macadamia nut oil, mango butter, manila oil, meadowfoam oil, mid chain tryglycerides liquid (mct), mineral oil, moringa seed oil, murumuru oil, Oenothera biennis oil, olive oil, orange oil, Orbignya oleifera seed oil, palm oil, pamergranet seed oil, papaya seed oil, paraffinum liquidum Persea gratissima oil, peach kernel oil, perilla oil, petrolatum, polyglycerin-3, primerose oil, pumpkin seed oil, Punica granatum seed oil, rasberry seed oil, rice bran oil, rosehip oil, safflower oil, sal butter, Schinziophyton rautanenil seed oil, sesame oil, shae butter oil, shorea (sal) butter oil, sorbitol, soybean oil, sunflower oil, squalene oil, tamanu oil, terpene oil, Theobroma grandiflorum seed butter, tomato seed oil, vegetable oil, walnut oil, and wheat germ ol. In some embodiments, the lip scrub composition comprises about 5% to about 15% w/w of the emollient.

In some embodiments, the emollient is green tea oil. In some embodiments, the lip scrub composition comprises at least about 1%, 3%, 5%, 7%, 8%, 9%, or 10% w/w of green tea oil. In some embodiments, the lip scrub composition comprises at least about 10% w/w of green tea oil. In some embodiments, the lip scrub composition comprises between about 1% to about 15%, about 5% to about 15%, or about 10% to about 15% w/w of green tea oil.

In some embodiments, the emollient is Camelina oil. In some embodiments, the lip scrub composition comprises at least about 1%, 3%, 5%, 7%, 8%, 9%, or 10% w/w of Camelina oil. In some embodiments, the lip scrub composition comprises at least about 10% w/w of Camelina oil. In some embodiments, the lip scrub composition comprises between about 1% to about 15%, about 5% to about 15%, or about 10% to about 15% w/w of Camelina oil.

In some embodiments, the emollient is cranberry seed oil. In some embodiments, the lip scrub composition comprises at least about 1%, 2%, 3%, 4%, or 5% w/w of cranberry seed oil. In some embodiments, the lip scrub composition comprises at least about 3% w/w of cranberry seed oil. In some embodiments, the lip scrub composition comprises between about 1% to about 5%, about 2% to about 5%, or about 2% to about 4% w/w of cranberry seed oil.

In some embodiments, the emollient is pumpkin seed oil. In some embodiments, the lip scrub composition comprises at least about 1%, 3%, 5%, 7%, 8%, 9%, or 10% w/w of pumpkin seed oil. In some embodiments, the lip scrub composition comprises at least about 10% w/w of pumpkin seed oil. In some embodiments, the lip scrub composition comprises between about 1% to about 15%, about 5% to about 15%, or about 10% to about 15% w/w of pumpkin seed oil.

In some embodiments, the emollient is grapeseed oil. In some embodiments, the lip scrub composition comprises at least about 1%, 2%, 3%, 4%, or 5% w/w of grapeseed oil. In some embodiments, the lip scrub composition comprises at least about 3% w/w of grapeseed oil. In some embodiments, the lip scrub composition comprises between about 1% to about 5%, about 2% to about 5%, or about 2% to about 4% w/w of grapeseed oil.

In some embodiments, the emollient is sal butter. In some embodiments, the lip scrub composition comprises at least about 1%, 2%, 3%, 4%, or 5% w/w of sal butter. In some embodiments, the lip scrub composition comprises at least about 3% w/w of sal butter. In some embodiments, the lip scrub composition comprises between about 1% to about 5%, about 2% to about 5%, or about 2% to about 4% w/w of sal butter.

In some embodiments, the lip scrub composition comprises a flavoring agent. In some embodiments, the flavoring agent is selected from: sugar-free powder flavor, sugar-free flavor oil, sugar-free extract flavor, sugar-free sweetener, green tea powder, and fine coffee ground. In some embodiments, the sugar-free flavor oil is selected from: acai flavor oil, almond flavor oil, amaretto cappuccino flavor oil, amaretto flavor oil, anise flavor oil, apple flavor oil, apple cider flavor oil, apple pie flavor oil, apple rosemary flavor oil, apricot flavor oil, bacon flavor oil, banana flavor oil, banana foster flavor oil, Bavarian cream flavor oil, berry flavor oil, blackberry flavor oil, black cherry flavor oil, black currant flavor oil, black raspberry flavor oil, blueberry flavor oil, bubble gum flavor oil, butter flavor oil, butter pecan flavor oil, butterscotch flavor oil, cake batter flavor oil, candy cane flavor oil, caramel flavor oil, cherry flavor oil, chocolate flavor oil, cinnamon flavor oil, coconut flavor oil, coffee flavor oil, cotton candy flavor oil, cupcake frosting flavor oil, grape flavor oil, grapefruit flavor oil, hazelnut flavor oil, honey flavor oil, key lime pie flavor oil, lavender flavor oil, lemon flavor oil, lime flavor oil, mango flavor oil, mint flavor oil, orange flavor oil, orange creamsicle flavor oil, papaya flavor oil, peach flavor oil, pear flavor oil, peppermint flavor oil, pina-coloda flavor oil, pomegranate flavor oil, raspberry flavor oil, rootbeer flavor oil, spearmint flavor oil, strawberry flavor oil, and watermelon flavor oil. In some embodiments, the sugar-free sweetener is stevia. In some embodiments, the lip scrub composition comprises about 1% to about 3% w/w of the flavoring agent.

In some embodiments, the flavoring agent is natural orange flavor oil. In some embodiments, the lip scrub composition comprises at least about 0.1%, 0.2%, 0.3%, 0.4%, or 0.5% w/w of natural orange flavor oil. In some embodiments, the lip scrub composition comprises at least about 0.2% w/w of natural orange flavor oil. In some embodiments, the lip scrub composition comprises between about 0.05% to about 0.4%, about 0.05% to about 0.3%, about 0.1% to about 0.3% w/w of natural orange flavor oil.

In some embodiments, the lip scrub composition comprises a thickening agent. In some embodiments, the thickening agent is selected from almond butter, aloe butter, argon butter, avocado butter, beeswax, candelilla wax, carnauba wax, chamomile, cocoa butter, coconut butter, coffee butter, carrot seed butter, cranberry butter, cupuacu butter, emulsifying wax, Euphorbia cerifera wax, grean tea butter, hemp butter, hempseed butter, horsetail butter, jojoba butter, jojoba wax, kokum butter, lanolin butter, lavender butter, lemon butter, macadamia butter, mango butter, olive butter, orange butter, ozokerite wax, sal butter, palm butter, polysorbate, pumpkin seed butter, shae butter, shorea (sal) butter, soy butter, Theobroma grandifiorum (Cupuacu) seed butter, tomato seed butter, tucuma butter, and tumeric butter. In some embodiments, the lip scrub composition comprises about 0% to about 15% w/w of the thickening agent. In some embodiments, the lip scrub composition comprises less than about 15%, 10%, 5%, or 1% w/w of shae butter. In some embodiments, the lip scrub composition does not comprise shae butter.

In some embodiments, the lip scrub composition comprises an essential oil. In some embodiments, the essential oil is selected from citral, grapefruit, lavender, lemon, lime, orange, peppermint, terpene oil, vanilla, and wintergreen. In some embodiments, the terpene oil is selected from pinene, myrcene, limonene, caryophyllene, linalool, humulene, ocimene, and terpinolene. In some embodiments, the lip scrub composition comprises about 0.5% to about 1% w/w of the essential oil. In some embodiments, the essential oil is a food grade essential oil.

In some embodiments, the lip scrub composition comprises a colorant. In some embodiments, the colorant is selected from food grade colorants powder or oil, sugar free colorants powder or oil, micas, pigment, and liquid dye. In some embodiments, the lip scrub composition comprises about 0% to about 1% w/w of the colorant.

In some embodiments, the lip scrub composition comprises a vitamin. In some embodiments, the vitamin is selected from vitamin C, vitamin E, and vitamin K1. In some embodiments, the lip scrub composition comprises about 0% to about 1% w/w of the vitamin. In some embodiments, the lip scrub composition does not comprise vitamin K1.

In some embodiments, the lip scrub composition comprises a humectant. In some embodiments, the humectant is selected from: Crambe abyssinica seed oil, glycerin, lecithin, and propylene glycol. In some embodiments, the lip scrub composition comprises about 0% to about 10% w/w of the humectant.

In some embodiments, the lip scrub composition comprises a preservative. In some embodiments, the preservative is selected from: benyzl alcohol, dehydroacetic acid, glyceryl caprilate, hydroxyacetophenone, potassium sorbate, caprylhydroxamic acid, caprylyl glycol, lycerin, geogard ECT, sensicare M4200, Geogard Ultra, anisic acid, glyceryl caprylate, glyceryl undecylenate, naticide, ehtyl lauroyl arginate, and biosecur. In some embodiments, the lip scrub composition comprises about 0.15% to 0.3% w/w of the preservative.

In some embodiments, the preservative is potassium sorbate. In some embodiments, the lip scrub composition comprises at least about 0.02%, 0.04%, 0.08%, 0.1%, 0.13%, 0.15%, 0.17%, 0.19%, 0.2%, 0.25%, or 0.3% w/w of potassium sorbate. In some embodiments, the lip scrub composition comprises at least about 0.15% w/w of potassium sorbate. In some embodiments, the lip scrub composition comprises between about 0.05% to about 0.2% about 0.1% to about 0.2%, about 0.13% to about 0.18% w/w of potassium sorbate.

In some embodiments, the lip scrub composition comprises an abrasive. In some embodiments, the abrasive is not a sugar alcohol. In some embodiments, abrasive is selected from sugar-free sprinkles nonpareil, fruit peel, fruit powder, vegetable peel, vegetable powder, and jojoba beads. In some embodiments, the fruit peel is selected from orange peel, grapefruit peel, carrot peel, pumpkin peel, and lemon peel. In some embodiments, the fruit peel is carrot peel. In some embodiments, the fruit peel is pumpkin peel. In some embodiments, the abrasive is not a sugar. In some embodiments, the lip scrub composition comprises about 0% to about 15% w/w of the abrasive.

In some embodiments, the lip scrub composition comprises less than about 95%, 90%, 80%, 70%, 60%, 50%, 40%, 30%, 20%, 10%, 9%, 8%, 7%, 6%, 5%, 4%, 3%, 2%, or 1% w/v water.

In some embodiments, the lip scrub composition further comprises a lip plumping agent. In some embodiments, the lip plumping agent is selected from sodium hyaluronate, cinnamon, capsicum, and menthol. In some embodiments, the lip plumping agent is sodium hyaluronate. In some embodiments, the lip scrub composition comprises about 0.01% to about 3% w/w of the lip plumping agent.

In some embodiments, the lip scrub composition further comprises a drug agent. In some embodiments, the drug agent is cannabidol (cbd) oil.

In some embodiments, the lip scrub composition is formulated as a lip balm, lip stick, lip cream, lip gel, or lip gloss. In some embodiments, the lip scrub composition is translucent. In some embodiments, a translucent lip scrub composition is free of added color or tint, such that when the translucent lip scrub composition is applied to the lips, it does not add color or tint to the lips. In some embodiments, the lip scrub composition is tinted. In some embodiments, a tinted lip scrub composition contains a colorant or tint that adds color or tint to the lips once the tinted lip scrub composition is applied to the lips.

Further disclosed herein is a lip scrub product comprising any of the lip scrub compositions disclosed herein. In some embodiments, the lip scrub composition is contained in a container of any shape or size. In some embodiments, the container is a jar or tube.

In some embodiments, the lip scrub product further comprises an applicator. In some embodiments, the applicator is used to apply the lip scrub composition to the lips.

Further disclosed herein is a method of exfoliating lips of a subject, the method comprising topically administering to the lips of the subject any of the lip scrub compositions disclosed herein. In some embodiments, the lip scrub composition is applied directly to the lips. In some embodiments, the lip scrub composition is applied to the lips via an applicator. In some embodiments, the lip scrub composition is rubbed onto the subject's fingers and then applied to the lips.

Further disclosed herein is a method of exfoliating lips of a subject, the method comprising topically administering to the lips of the subject any of the lip scrub products disclosed herein. In some embodiments, the lip scrub composition is applied directly to the lips. In some embodiments, the lip scrub composition is applied to the lips via an applicator. In some embodiments, the lip scrub composition is rubbed onto the subject's fingers and then applied to the lips.

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### BRIEF DESCRIPTION OF THE DRAWINGS

FIGS. 1A-1F illustrate exemplary containers for holding the lip scrub composition disclosed herein.

### DETAILED DESCRIPTION OF THE INVENTION

Disclosed herein are sugar-free lip scrub compositions. In some embodiments, a lip scrub composition comprises an exfoliating agent that is a granulated sugar alcohol, wherein the lip scrub composition comprises less than about 95%, 90%, 80%, 70%, 60%, 50%, 40%, 30%, 20%, or 10% w/w sugar.

In some embodiments, a lip scrub composition comprises (a) one or more exfoliating agents; (b) one or more emollients; (c) one or more flavoring agents; (d) one or more colorants; and (e) one or more preservatives. In some embodiments, the exfoliating agent is a granulated sugar alcohol. In some embodiments, the exfoliating agent is not a granulated sugar.

In some embodiments, a lip scrub composition comprises (a) one or more exfoliating agents; (b) one or more emollients; (c) one or more abrasives; (d) one or more flavoring agents; (e) one or more colorants; and (f) one or more preservatives. In some embodiments, the exfoliating agent is a granulated sugar alcohol. In some embodiments, the exfoliating agent is not a granulated sugar.

In some embodiments, a lip scrub composition comprises (a) one or more exfoliating agents; (b) one or more emollients; (c) one or more abrasives; (d) one or more flavoring agents; (e) one or more colorants; (f) one or more drug agents; and (g) one or more preservatives. In some embodiments, the exfoliating agent is a granulated sugar alcohol. In some embodiments, the exfoliating agent is not a granulated sugar.

In some embodiments, any of the lip scrub compositions disclosed herein are edible. In some embodiments, any of the lip scrub compositions disclosed herein are safe for human consumption.

Exfoliating Agent

Any of the lip scrub compositions disclosed herein may comprise one or more exfoliating agents, wherein the exfoliating agent is not a sugar. In some embodiments, any of the lip scrub compositions disclosed herein comprise 1, 2, 3, 4, or 5 or more exfoliating agents.

In some embodiments, any of the lip scrub compositions disclosed herein comprise 1, 2, 3, 4, or 5 or more exfoliating agents, wherein at least one exfoliating is not a sugar. Non-limiting examples of sugars include, but are not limited to, brown sugar and white sugar. In some embodiments, the sugar comprises sucrose. In some embodiments, the sugar is a granulated sugar. In some embodiments, any of the lip scrub compositions disclosed herein do not comprise an exfoliating agent that is a granulated sugar. In some embodiments, any of the lip scrub compositions disclosed herein do not comprise a granulated sugar. In some embodiments, any of the lip scrub compositions disclosed herein do not comprise brown sugar. In some embodiments, any of the lip scrub compositions disclosed herein do not comprise white sugar. In some embodiments, any of the lip scrub compositions disclosed herein do not comprise sucrose.

In some embodiments, any of the lip scrub compositions disclosed herein comprise 1, 2, 3, 4, or 5 or more exfoliating agents, wherein at least one exfoliating agent is a sugar alcohol. In some embodiments, the sugar alcohol is granulated. In some embodiments, the sugar alcohol is selected from erythritol, xylitol, mannitol, sorbitol, lactitol, isomalt, maltitol and hydrogenated starch hydrolysates (HSH).

In some embodiments, any of the lip scrub composition disclosed herein comprise at least about 40%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% w/w of the exfoliating agent(s). In some embodiments, any of the lip scrub compositions disclosed herein comprise at least about 50% w/w of the exfoliating agent(s). In some embodiments, any of the lip scrub compositions disclosed herein comprise at least about 60% w/w of the exfoliating agent(s). In some embodiments, any of the lip scrub compositions disclosed herein comprise at least about 70% w/w of the exfoliating agent(s). In some embodiments, any of the lip scrub compositions disclosed herein comprise at least about 75% w/w of the exfoliating agent(s). In some embodiments, any of the lip scrub compositions disclosed herein comprise at least about 80% w/w of the exfoliating agent(s). In some embodiments, any of the lip scrub compositions disclosed herein comprise at least about 81% w/W of the exfoliating agent(s). In some embodiments, any of the lip scrub compositions disclosed herein comprise at least about 82% w/w of the exfoliating agent(s). In some embodiments, any of the lip scrub compositions disclosed herein comprise at least about 83% w/w of the exfoliating agent(s).

In some embodiments, any of the lip scrub compositions disclosed herein comprise about 50% to about 90% w/w of the exfoliating agent(s). In some embodiments, any of the lip scrub compositions disclosed herein comprise about 60% to about 90% w/w of the exfoliating agent(s). In some embodiments, any of the lip scrub compositions disclosed herein comprise about 65% to about 90% w/w of the exfoliating agent(s). In some embodiments, any of the lip scrub compositions disclosed herein comprise about 70% to about 90% w/w of the exfoliating agent(s). In some embodiments, any of the lip scrub compositions disclosed herein comprise about 70% to about 90% w/w of the exfoliating agent(s). In some embodiments, any of the lip scrub compositions disclosed herein comprise about 75% to about 95% w/w of the exfoliating agent(s). In some embodiments, any of the lip scrub compositions disclosed herein comprise about 70% to about 95% w/w of the exfoliating agent(s). In some embodiments, any of the lip scrub compositions disclosed herein comprise about 65% to about 95% w/w of the exfoliating agent(s). In some embodiments, any of the lip scrub compositions disclosed herein comprise about 77% to about 95% w/w of the exfoliating agent(s). In some embodiments, any of the lip scrub compositions disclosed herein comprise about 80% to about 95% w/w of the exfoliating agent(s). In some embodiments, any of the lip scrub compositions disclosed herein comprise about 75% to about 85% w/w of the exfoliating agent(s). In some embodiments, any of the lip scrub compositions disclosed herein comprise about 70% to about 85% w/w of the exfoliating agent(s). In some embodiments, any of the lip scrub compositions disclosed herein comprise about 65% to about 85% w/w of the exfoliating agent(s). In some embodiments, any of the lip scrub compositions disclosed herein comprise about 77% to about 85% w/w of the exfoliating agent(s). In some embodiments, any of the lip scrub compositions disclosed herein comprise about 80% to about 85% w/w of the exfoliating agent(s).

In some embodiments, the exfoliating agent is erythritol. In some embodiments, any of the lip scrub compositions disclosed herein comprise at least about 70%, 75%, 80%, 81%, 82%, 83%, 84%, 85%, 86%, 87%, 88%, 89%, 90%, 91%, 92%, 93%, 94%, 95%, 96%, 97%, 98%, or 99% w/w of erythritol. In some embodiments, any of the lip scrub compositions disclosed herein comprise at least about 80% w/w of erythritol. In some embodiments, any of the lip scrub compositions disclosed herein comprise at least about 81% w/w of erythritol. In some embodiments, any of the lip scrub compositions disclosed herein comprise at least about 85% w/w of erythritol. In some embodiments, any of the lip scrub compositions disclosed herein comprise at least about 87% w/w of erythritol. In some embodiments, any of the lip scrub compositions disclosed herein comprise at least about 90% w/w of erythritol. In some embodiments, any of the lip scrub compositions disclosed herein comprise about 70% to about 90% w/w of the erythritol. In some embodiments, any of the lip scrub compositions disclosed herein comprise about 70% to about 90% w/w of the erythritol. In some embodiments, any of the lip scrub compositions disclosed herein comprise about 75% to about 85% w/w of the erythritol. In some embodiments, any of the lip scrub compositions disclosed herein comprise about 70% to about 85% w/w of the erythritol. In some embodiments, any of the lip scrub compositions disclosed herein comprise about 65% to about 85% w/w of the erythritol. In some embodiments, any of the lip scrub compositions disclosed herein comprise about 77% to about 85% w/w of the erythritol. In some embodiments, any of the lip scrub compositions disclosed herein comprise about 80% to about 85% w/w of the erythritol. In some embodiments, any of the lip scrub compositions disclosed herein comprise about 70% to about 95% w/w of the erythritol. In some embodiments, any of the lip scrub compositions disclosed herein comprise about 65% to about 95% w/w of the erythritol. In some embodiments, any of the lip scrub compositions disclosed herein comprise about 77% to about 95% w/w of the erythritol. In some embodiments, any of the lip scrub compositions disclosed herein comprise about 80% to about 95% w/w of the erythritol.

In some embodiments, the exfoliating agent is not Citrus Limon peel powder. In some embodiments, any of the lip scrub compositions disclosed herein comprise less than about 95%, 90%, 80%, 70%, 60%, 50%, 40%, 30%, 25%, 20%, 15%, 10%, 9%, 8%, 7%, 6%, 5%, 4%, 3%, 2%, or 1% w/w Citrus limon peel powder, In some embodiments, any of the lip scrub compositions disclosed herein comprise less than about 30% w/w Citrus limon peel powder. In some embodiments, any of the lip scrub compositions disclosed herein comprise less than about 20% w/w Citrus limon peel powder. In some embodiments, any of the lip scrub compositions disclosed herein comprise less than about 10% w/w Citrus limon peel powder. In some embodiments, any of the lip scrub compositions disclosed herein comprise less than about 5% w/w Citrus limon peel powder. In some embodiments, any of the lip scrub compositions disclosed herein comprise less than about 3% w/w Citrus limon peel powder. In some embodiments, any of the lip scrub compositions disclosed herein comprise less than about 1% w/w Citrus limon peel powder.

In some embodiments, the exfoliating agent is not Oryza Sativa rice powder. In some embodiments, any of the lip scrub compositions disclosed herein comprise less than about 90%, 80%, 70%, 60%, 50%, 40%, 30%, 25%, 20%, 15%, 10%, 9%, 8%, 7%, 6%, 5%, 4%, 3%, 2%, or 1% w/w Oryza saliva rice powder. In some embodiments, any of the lip scrub compositions disclosed herein comprise less than about 30% w/w Oryza sativa rice powder. In some embodiments, any of the lip scrub compositions disclosed herein comprise less than about 20% w/w Oryza sativa rice powder. In some embodiments, any of the lip scrub compositions disclosed herein comprise less than about 10% w/w Oryza sativa rice powder. In some embodiments, any of the lip scrub compositions disclosed herein comprise less than about 5% w/w Oryza sativa rice powder. In some embodiments, any of the lip scrub compositions disclosed herein comprise less than about 3% w/w Oryza sativa rice powder. In some embodiments, any of the lip scrub compositions disclosed herein comprise less than about 1% w/w Oryza saliva rice powder.

In some embodiments, the exfoliating agent is not allulose. In some embodiments, the lip scrub composition comprises less than about 90%, 80%, 70%, 60%, 50%, 40%, 30%, 25%, 20%, 15%, 10%, 9%, 8%, 7%, 6%, 5%, 4%, 3%, 2%, or 1% w/w allulose. In some embodiments, any of the lip scrub compositions disclosed herein comprise less than about 30% w/w Allulose powder. In some embodiments, any of the lip scrub compositions disclosed herein comprise less than about 20% w/w Allulose powder. In some embodiments, any of the lip scrub compositions disclosed herein comprise less than about 10% w/w Allulose powder. In some embodiments, any of the lip scrub compositions disclosed herein comprise less than about 5% w/w Allulose powder. In some embodiments, any of the lip scrub compositions disclosed herein comprise less than about 3% w/w Allulose powder. In some embodiments, any of the lip scrub compositions disclosed herein comprise less than about 1% w/w Allulose powder.

Emollient

In some embodiments, any of the lip scrub compositions disclosed herein comprise an emollient. In some embodiments, any of the lip scrub compositions disclosed herein comprise 1, 2, 3, 4, or 5 or more emollients. In some embodiments, any of the lip scrub compositions disclosed herein comprise 2 or more emollients. In some embodiments, any of the lip scrub compositions disclosed herein comprise 3 or more emollients.

In some embodiments, the emollient is selected from: Aloe vera oil, aprikot kernel oil, argan oil, avocado oil, babassu oil, blueberry seed oil, broccoli seed oil, Butyrospermum parkii unsaponifiables, Camellia kissi seed oil castor oil, canola oil, carrot seed oil, castor oil, celery seed oil, coconut oil, Cocos nucifera (Coconut) Oil, cucumber seed oil, dimethicone, flaxseed oil, glycerin, grapeseed oil, green tea oil, hazelnut oil, Helianthus animus seed oil, hemp oil, hydrogenated moringa oil esters, jojoba oil, lanolin, lavender oil, macadamia nut oil, mango butter, manila oil, meadowfoam oil, mid chain tryglycerides liquid (mct), mineral oil, moringa seed oil, murumuru oil, Oenothera biennis oil, olive oil, orange oil, Orbignya oleifera seed oil, palm oil, pamergranet seed oil, papaya seed oil, paraffinum liquidum Persea gratissima oil, peach kernel oil, perilla oil, petrolatum, polyglycerin-3, primerose oil, pumpkin seed oil, Punica granatum seed oil, rasberry seed oil, rice bran oil, rosehip oil, safflower oil, sal butter, Schinziophyton rautanenil seed oil, sesame oil, shae butter oil, shorea (sal) butter oil, sorbitol, soybean oil, sunflower oil, squalene oil, tamanu oil, terpene oil, Theobroma grandiflorum seed butter, tomato seed oil, vegetable oil, walnut oil, and wheat germ ol. In some embodiments, the emollient is green tea oil. In some embodiments, the emollient is pumpkin seed oil. In some embodiments, the emollient is grapeseed oil. In some embodiments, the emollient is sal butter.

In some embodiments, any of the lip scrub compositions disclosed herein comprise about 1% to about 30% w/w of the emollient(s). In some embodiments, any of the lip scrub compositions disclosed herein comprise about 1% to about 25% w/w of the emollient(s). In some embodiments, any of the lip scrub compositions disclosed herein comprise about 5% to about 20% w/w of the emollient(s). In some embodiments, any of the lip scrub compositions disclosed herein comprise about 1% to about 15% w/w of the emollient(s). In some embodiments, any of the lip scrub compositions disclosed herein comprise about 5% to about 30% w/w of the emollient(s). In some embodiments, any of the lip scrub compositions disclosed herein comprise about 5% to about 25% w/w of the emollient(s). In some embodiments, any of the lip scrub compositions disclosed herein comprise about 5% to about 20% w/w of the emollient(s). In some embodiments, any of the lip scrub compositions disclosed herein comprise about 5% to about 15% w/w of the emollient(s). In some embodiments, any of the lip scrub compositions disclosed herein comprise about 5% to about 10% w/w of the emollient(s).

In some embodiments, any of the lip scrub compositions disclosed herein comprise at least about 1%, 2%, 3%, 4%, 5%, 6%, 7%, 8%, 9%, 10%, 11%, 12%, 13%, 14%, or 15% w/w of the emollient(s). In some embodiments, any of the lip scrub compositions disclosed herein comprise at least about 1% w/w of the emollient(s). In some embodiments, any of the lip scrub compositions disclosed herein comprise at least about 5% w/w of the emollient(s). In some embodiments, any of the lip scrub compositions disclosed herein comprise at least about 10% w/w of the emollient(s). In some embodiments, any of the lip scrub compositions disclosed herein comprise at least about 15% w/w of the emollient(s).

Flavoring Agent

In some embodiments, any of the lip scrub compositions disclosed herein comprise a flavoring agent. In some embodiments, any of the lip scrub compositions disclosed herein comprise 1, 2, 3, 4, or 5 or more flavoring agents. In some embodiments, any of the lip scrub compositions disclosed herein comprise 2 or more flavoring agents. In some embodiments, any of the lip scrub compositions disclosed herein comprise 3 or more flavoring agents.

In some embodiments, the flavoring agent is selected from: sugar-free powder flavor, sugar-free flavor oil, sugar-free extract flavor, sugar-free sweetener, green tea powder, and fine coffee ground. In some embodiments, the sugar-free flavor oil is selected from: acai flavor oil, almond flavor oil, amaretto cappuccino flavor oil, amaretto flavor oil, anise flavor oil, apple flavor oil, apple cider flavor oil, apple pie flavor oil, apple rosemary flavor oil, apricot flavor oil, bacon flavor oil, banana flavor oil, banana foster flavor oil, Bavarian cream flavor oil, berry flavor oil, blackberry flavor oil, black cherry flavor oil, black currant flavor oil, black raspberry flavor oil, blueberry flavor oil, bubble gum flavor oil, butter flavor oil, butter pecan flavor oil, butterscotch flavor oil, cake batter flavor oil, candy cane flavor oil, caramel flavor oil, cherry flavor oil, chocolate flavor oil, cinnamon flavor oil, coconut flavor oil, coffee flavor oil, cotton candy flavor oil, cupcake frosting flavor oil, grape flavor oil, grapefruit flavor oil, hazelnut flavor oil, honey flavor oil, key lime pie flavor oil, lavender flavor oil, lemon flavor oil, lime flavor oil, mango flavor oil, mint flavor oil, orange flavor oil, orange creamsicle flavor oil, papaya flavor oil, peach flavor oil, pear flavor oil, peppermint flavor oil, pina-coloda flavor oil, pomegranate flavor oil, raspberry flavor oil, rootbeer flavor oil, spearmint flavor oil, strawberry flavor oil, and watermelon flavor oil. In some embodiments, the sugar-fee sweetner is stevia.

In some embodiments, any of the lip scrub compositions disclosed herein comprise about 0.1% to about 15% w/w of the flavoring agent(s). In some embodiments, any of the lip scrub compositions disclosed herein comprise about 0.5% to about 15% w/w of the flavoring agent(s). In some embodiments, any of the lip scrub compositions disclosed herein comprise about 1% to about 15% w/w of the flavoring agent(s). In some embodiments, any of the lip scrub compositions disclosed herein comprise about 0.1% to about 10% w/w of the flavoring agent(s). In some embodiments, any of the lip scrub compositions disclosed herein comprise about 0.5% to about 15% w/w of the flavoring agent(s). In some embodiments, any of the lip scrub compositions disclosed herein comprise about 1% to about 10% w/w of the flavoring agent(s). In some embodiments, any of the lip scrub compositions disclosed herein comprise about 0.1% to about 5% w/w of the flavoring agent(s). In some embodiments, any of the lip scrub compositions disclosed herein comprise about 0.5% to about 5% w/w of the flavoring agent(s). In some embodiments, any of the lip scrub compositions disclosed herein comprise about 1% to about 5% w/w of the flavoring agent(s). In some embodiments, any of the lip scrub compositions disclosed herein comprise about 0.1% to about 4% w/w of the flavoring agent(s). In some embodiments, any of the lip scrub compositions disclosed herein comprise about 0.5% to about 4% w/w of the flavoring agent(s). In some embodiments, any of the lip scrub compositions disclosed herein comprise about 1% to about 4% w/w of the flavoring agent(s). In some embodiments, any of the lip scrub compositions disclosed herein comprise about 0.1% to about 3% w/w of the flavoring agent(s). In some embodiments, any of the lip scrub compositions disclosed herein comprise about 0.5% to about 3% w/w of the flavoring agent(s). In some embodiments, any of the lip scrub compositions disclosed herein comprise about 1% to about 3% w/w of the flavoring agent(s).

In some embodiments, any of the lip scrub compositions disclosed herein comprise at least about 0.1%, 0.2%, 0.3%, 0.4%, 0.5%, 0.6%, 0.7%, 0.8%, 0.9%, 1%, 1.5%, 2%, 2.5%, or 3% w/w of the flavoring agent(s). In some embodiments, any of the lip scrub compositions disclosed herein comprise at least about 0.1% w/w of the flavoring agent(s). In some embodiments, any of the lip scrub compositions disclosed herein comprise at least about 0.5% w/w of the flavoring agent(s). In some embodiments, any of the lip scrub compositions disclosed herein comprise at least about 1% w/w of the flavoring agent(s). In some embodiments, any of the lip scrub compositions disclosed herein comprise at least about 2% w/w of the flavoring agent(s). In some embodiments, any of the lip scrub compositions disclosed herein comprise at least about 3% w/w of the flavoring agent(s).

In some embodiments, the flavoring agent is stevia. In some embodiments, any of the lip scrub compositions disclosed herein comprise at least about 0.1%, 0.2%, 0.3%, 0.4%, 0.5%, 0.6%, 0.7%, 0.8%, 0.9%, 1%, 1.5%, 2%, 2.5%, or 3% w/w of stevia. In some embodiments, any of the lip scrub compositions disclosed herein comprise at least about 0.1% w/w of stevia. In some embodiments, any of the lip scrub compositions disclosed herein comprise at least about 0.5% w/w of stevia. In some embodiments, any of the lip scrub compositions disclosed herein comprise at least about 1% w/w of stevia. In some embodiments, any of the lip scrub compositions disclosed herein comprise at least about 2% w/w of stevia. In some embodiments, any of the lip scrub compositions disclosed herein comprise at least about 3% w/w of stevia.

Thickening Agent

In some embodiments, any of the lip scrub compositions disclosed herein comprise a thickening agent. In some embodiments, any of the lip scrub compositions disclosed herein comprise 1, 2, 3, 4, or 5 or more thickening agents. In some embodiments, any of the lip scrub compositions disclosed herein comprise 2 or more thickening agents. In some embodiments, any of the lip scrub compositions disclosed herein comprise 3 or more thickening agents.

In some embodiments, any of the lip scrub compositions disclosed herein comprise a thickening agent. In some embodiments, the thickening agent is selected from almond butter, aloe butter, argon butter, avocado butter, beeswax, candelilla wax, carnauba wax, chamomile, cocoa butter, coconut butter, coffee butter, carrot seed butter, cranberry butter, cupuacu butter, emulsifying wax, Euphorbia cerifera wax, grean tea butter, hemp butter, hempseed butter, horsetail butter, jojoba butter, jojoba wax, kokum butter, lanolin butter, lavender butter, lemon butter, macadamia butter, mango butter, olive butter, orange butter, ozokerite wax, sal butter, palm butter, polysorbate, pumpkin seed butter, shae butter, shorea (sal) butter, soy butter, Theobroma grandifiorum (Cupuacu) seed butter, tomato seed butter, tucuma butter, and tumeric butter.

In some embodiments, any of the lip scrub compositions disclosed herein comprise about 0% to about 15% w/w of the thickening agent. In some embodiments, any of the lip scrub compositions disclosed herein comprise about 1% to about 30% w/w of the thickening agent(s). In some embodiments, any of the lip scrub compositions disclosed herein comprise about 1% to about 25% w/w of the thickening agent(s). In some embodiments, any of the lip scrub compositions disclosed herein comprise about 5% to about 20% w/w of the thickening agent(s). In some embodiments, any of the lip scrub compositions disclosed herein comprise about 1% to about 15% w/w of the thickening agent(s). In some embodiments, any of the lip scrub compositions disclosed herein comprise about 5% to about 30% w/w of the thickening agent(s). In some embodiments, any of the lip scrub compositions disclosed herein comprise about 5% to about 25% w/w of the thickening agent(s). In some embodiments, any of the lip scrub compositions disclosed herein comprise about 5% to about 20% w/w of the thickening agent(s). In some embodiments, any of the lip scrub compositions disclosed herein comprise about 5% to about 15% w/w of the thickening agent(s). In some embodiments, any of the lip scrub compositions disclosed herein comprise about 5% to about 10% w/w of the thickening agent(s).

In some embodiments, any of the lip scrub compositions disclosed herein comprise at least about 1%, 2%, 3%, 4%, 5%, 6%, 7%, 8%, 9%, 10%, 11%, 12%, 13%, 14%, or 15% w/w of the thickening agent(s). In some embodiments, any of the lip scrub compositions disclosed herein comprise at least about 1% w/w of the thickening agent(s). In some embodiments, any of the lip scrub compositions disclosed herein comprise at least about 5% w/w of the thickening agent(s). In some embodiments, any of the lip scrub compositions disclosed herein comprise at least about 10% w/w of the thickening agent(s). In some embodiments, any of the lip scrub compositions disclosed herein comprise at least about 15% w/w of the thickening agent(s).

In some embodiments, any of the lip scrub compositions disclosed herein comprise less than about 15%, 14%, 13%, 12%, 11%, 10%, 9%, 8%, 7%, 6%, 5%, 4%, 3%, 2%, or 1% w/w of thickening agent(s). In some embodiments, any of the lip scrub compositions disclosed herein comprise less than about 10% w/w of thickening agent(s). In some embodiments, any of the lip scrub compositions disclosed herein comprise less than about 5% w/w of thickening agent(s). In some embodiments, any of the lip scrub compositions disclosed herein comprise less than about 1% w/w of thickening agent(s). In some embodiments, any of the lip scrub compositions disclosed herein do not comprise any thickening agent(s).

In some embodiments, any of the lip scrub compositions disclosed herein comprise less than about 15%, 14%, 13%, 12%, 11%, 10%, 9%, 8%, 7%, 6%, 5%, 4%, 3%, 2%, or 1% w/w of shae butter. In some embodiments, any of the lip scrub compositions disclosed herein comprise less than about 10% w/w of shae butter. In some embodiments, any of the lip scrub compositions disclosed herein comprise less than about 5% w/w of shae butter. In some embodiments, any of the lip scrub compositions disclosed herein comprise less than about 1% w/w of shae butter. In some embodiments, any of the lip scrub compositions disclosed herein do not comprise shae butter.

Essential Oil

In some embodiments, any of the lip scrub compositions disclosed herein comprise an essential oil. In some embodiments, any of the lip scrub compositions disclosed herein comprise 1, 2, 3, 4. or 5 or more essential oils. In some embodiments, any of the lip scrub compositions disclosed herein comprise 2 or more essential oils. In some embodiments, any of the lip scrub compositions disclosed herein comprise 3 or more essential oils.

In some embodiments, any of the lip scrub compositions disclosed herein comprise an essential oil. In some embodiments, the essential oil is selected from citral, grapefruit, lavender, lemon, lime, orange, peppermint, terpene oil, vanilla, and wintergreen. In some embodiments, the terpene oil is selected from pinene, myrcene, limonene, caryophyllene,linalool, humulene, ocimene, and terpinolene. In some embodiments, the essential oil is a food grade essential oil.

In some embodiments, any of the lip scrub compositions disclosed herein comprise about 0% to about 10% w/w of the essential oil(s). In some embodiments, any of the lip scrub compositions disclosed herein comprise about 0.1% to about 10% w/w of the essential oil(s). In some embodiments, any of the lip scrub compositions disclosed herein comprise about 0.2% to about 10% w/w of the essential oil(s). In some embodiments, any of the lip scrub compositions disclosed herein comprise about 0.5% to about 10% w/w of the essential oil(s). In some embodiments, any of the lip scrub compositions disclosed herein comprise about 0% to about 7% w/w of the essential oil(s). In some embodiments, any of the lip scrub compositions disclosed herein comprise about 0% to about 5% w/w of the essential oil(s). In some embodiments, any of the lip scrub compositions disclosed herein comprise about 0% to about 3% w/w of the essential oil(s). In some embodiments, any of the lip scrub compositions disclosed herein comprise about 0% to about 2% w/w of the essential oil(s). In some embodiments, any of the lip scrub compositions disclosed herein comprise about 0% to about 1% w/w of the essential oil(s). In some embodiments, any of the lip scrub compositions disclosed herein comprise about 0.1% to about 7% w/w of the essential oil(s). In some embodiments, any of the lip scrub compositions disclosed herein comprise about 0.1% to about 5% w/w of the essential oil(s). In some embodiments, any of the lip scrub compositions disclosed herein comprise about 0.1% to about 3% w/w of the essential oil(s). In some embodiments, any of the lip scrub compositions disclosed herein comprise about 0.1% to about 2% w/w of the essential oil(s). In some embodiments, any of the lip scrub compositions disclosed herein comprise about 0.1% to about 1% w/w of the essential oil(s). In some embodiments, any of the lip scrub compositions disclosed herein comprise about 0.3% to about 7% w/w of the essential oil(s). In some embodiments, any of the lip scrub compositions disclosed herein comprise about 0.3% to about 5% w/w of the essential oil(s). In some embodiments, any of the lip scrub compositions disclosed herein comprise about 0.3% to about 3% w/w of the essential oil(s). In some embodiments, any of the lip scrub compositions disclosed herein comprise about 0.3% to about 2% w/w of the essential oil(s). In some embodiments, any of the lip scrub compositions disclosed herein comprise about 0.3% to about 1% w/w of the essential oil(s). In some embodiments, any of the lip scrub compositions disclosed herein comprise about 0.5% to about 7% w/w of the essential oil(s). In some embodiments, any of the lip scrub compositions disclosed herein comprise about 0.5% to about 5% w/w of the essential oil(s). In some embodiments, any of the lip scrub compositions disclosed herein comprise about 0.5% to about 3% w/w of the essential oil(s). In some embodiments, any of the lip scrub compositions disclosed herein comprise about 0.5% to about 2% w/w of the essential oil(s). In some embodiments, any of the lip scrub compositions disclosed herein comprise about 0.5% to about 1% w/w of the essential oil(s).

In some embodiments, any of the lip scrub compositions disclosed herein comprise at least about 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, or 2% w/w of the essential oil(s). In some embodiments, any of the lip scrub compositions disclosed herein comprise at least about 0.1% w/w of the essential oil(s). In some embodiments, any of the lip scrub compositions disclosed herein comprise at least about 0.2% w/w of the essential oil(s). In some embodiments, any of the lip scrub compositions disclosed herein comprise at least about 0.3% w/w of the essential oil(s). In some embodiments, any of the lip scrub compositions disclosed herein comprise at least about 0.5% w/w of the essential oil(s). In some embodiments, any of the lip scrub compositions disclosed herein comprise at least about 0.7% w/w of the essential oil(s). In some embodiments, any of the lip scrub compositions disclosed herein comprise at least about 1% w/w of the essential oil(s).

Colorant

In some embodiments, any of the lip scrub compositions disclosed herein comprise a colorant. In some embodiments, any of the lip scrub compositions disclosed herein comprise 1, 2, 3, 4, or 5 or more colorants. In some embodiments, any of the lip scrub compositions disclosed herein comprise 2 or more colorants. In some embodiments, any of the lip scrub compositions disclosed herein comprise 3 or more colorants.

In some embodiments, the lip scrub composition comprises a colorant. In some embodiments, the colorant is selected from food grade colorants powder or oil, sugar free colorants powder or oil, micas, pigment, starch, and liquid dye. In some embodiments, the colorant is orange (propylene glycol lecithin, yellow 6 lake). In some embodiments, the colorant is yellow (propylene glycol lecithin, yellow 5 lake). In some embodiments, the colorant is green (propolyne glycol lecithin, yellow 5 lake, blue 1). In some embodiments, the colorant is titanium dioxide. In some embodiments, any of the lip scrub compositions disclosed herein do not comprise titanium dioxide. In some embodiments, the colorant is a starch. In some embodiments, the starch is a rice starch. In some embodiments, the rice starch is Oryza sativa rice.

In some embodiments, any of the lip scrub compositions disclosed herein comprise about 0% to about 10% w/w of the colorant(s). In some embodiments, any of the lip scrub compositions disclosed herein comprise about 0.1% to about 10% w/w of the colorant(s). In some embodiments, any of the lip scrub compositions disclosed herein comprise about 0.2% to about 10% w/w of the colorant(s). In some embodiments, any of the lip scrub compositions disclosed herein comprise about 0.5% to about 10% w/w of the colorant(s). In some embodiments, any of the lip scrub compositions disclosed herein comprise about 0% to about 7% w/w of the colorant(s). In some embodiments, any of the lip scrub compositions disclosed herein comprise about 0% to about 5% w/w of the colorant(s). In some embodiments, any of the lip scrub compositions disclosed herein comprise about 0% to about 3% w/w of the colorant(s). In some embodiments, any of the lip scrub compositions disclosed herein comprise about 0% to about 2% w/w of the colorant(s). In some embodiments, any of the lip scrub compositions disclosed herein comprise about 0% to about 1% w/w of the colorant(s). In some embodiments, any of the lip scrub compositions disclosed herein comprise about 0.1% to about 7% w/w of the colorant(s). In some embodiments, any of the lip scrub compositions disclosed herein comprise about 0.1% to about 5% w/w of the colorant(s). In some embodiments, any of the lip scrub compositions disclosed herein comprise about 0.1% to about 3% w/w of the colorant(s). In some embodiments, any of the lip scrub compositions disclosed herein comprise about 0.1% to about 2% w/w of the colorant(s). In some embodiments, any of the lip scrub compositions disclosed herein comprise about 0.1% to about 1% w/w of the colorant(s). In some embodiments, any of the lip scrub compositions disclosed herein comprise about 0.3% to about 7% w/w of the colorant(s). In some embodiments, any of the lip scrub compositions disclosed herein comprise about 0.3% to about 5% w/w of the colorant(s). In some embodiments, any of the lip scrub compositions disclosed herein comprise about 0.3% to about 3% w/w of the colorant(s). In some embodiments, any of the lip scrub compositions disclosed herein comprise about 0.3% to about 2% w/w of the colorant(s). In some embodiments, any of the lip scrub compositions disclosed herein comprise about 0.3% to about 1% w/w of the colorant(s). In some embodiments, any of the lip scrub compositions disclosed herein comprise about 0.5% to about 7% w/w of the colorant(s). In some embodiments, any of the lip scrub compositions disclosed herein comprise about 0.5% to about 5% w/w of the colorant(s). In some embodiments, any of the lip scrub compositions disclosed herein comprise about 0.5% to about 3% w/w of the colorant(s). In some embodiments, any of the lip scrub compositions disclosed herein comprise about 0.5% to about 2% w/w of the colorant(s). In some embodiments, any of the lip scrub compositions disclosed herein comprise about 0.5% to about 1% w/w of the colorant(s).

In some embodiments, any of the lip scrub compositions disclosed herein comprise at least about 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, or 2% w/w of the colorant(s). In some embodiments, any of the lip scrub compositions disclosed herein comprise at least about 0.1% w/w of the colorant(s). In some embodiments, any of the lip scrub compositions disclosed herein comprise at least about 0.2% w/w of the colorant(s). In some embodiments, any of the lip scrub compositions disclosed herein comprise at least about 0.3% w/w of the colorant(s). In some embodiments, any of the lip scrub compositions disclosed herein comprise at least about 0.5% w/w of the colorant(s). In some embodiments, any of the lip scrub compositions disclosed herein comprise at least about 0.7% w/w of the colorant(s). In some embodiments, any of the lip scrub compositions disclosed herein comprise at least about 1% w/w of the colorant(s).

In some embodiments, any of the lip scrub compositions disclosed herein comprise at least about 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, or 2% w/w of orange (propylene glycol lecithin, yellow 6 lake). In some embodiments, any of the lip scrub compositions disclosed herein comprise at least about 0.1% w/w of orange (propylene glycol lecithin, yellow 6 lake). In some embodiments, any of the lip scrub compositions disclosed herein comprise at least about 0.2% w/w of orange (propylene glycol lecithin, yellow 6 lake). In some embodiments, any of the lip scrub compositions disclosed herein comprise at least about 0.3% w/w of orange (propylene glycol lecithin, yellow 6 lake). In some embodiments, any of the lip scrub compositions disclosed herein comprise at least about 0.5% w/w of orange (propylene glycol lecithin, yellow 6 lake). In some embodiments, any of the lip scrub compositions disclosed herein comprise at least about 0.7% w/w of orange (propylene glycol lecithin, yellow 6 lake). In some embodiments, any of the lip scrub compositions disclosed herein comprise at least about 1% w/w of orange (propylene glycol lecithin, yellow 6 lake).

In some embodiments, any of the lip scrub compositions disclosed herein comprise at least about 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, or 2% w/w of yellow (propylene glycol lecithin, yellow 5 lake). In some embodiments, any of the lip scrub compositions disclosed herein comprise at least about 0.1% w/w of yellow (propylene glycol lecithin, yellow 5 lake). In some embodiments, any of the lip scrub compositions disclosed herein comprise at least about 0.2% w/w of yellow (propylene glycol lecithin, yellow 5 lake). In some embodiments, any of the lip scrub compositions disclosed herein comprise at least about 0.3% w/w of yellow (propylene glycol lecithin, yellow 5 lake). In some embodiments, any of the lip scrub compositions disclosed herein comprise at least about 0.5% w/w of yellow (propylene glycol lecithin, yellow 5 lake). In some embodiments, any of the lip scrub compositions disclosed herein comprise at least about 0.7% w/w of yellow (propylene glycol lecithin, yellow 5 lake). In some embodiments, any of the lip scrub compositions disclosed herein comprise at least about 1% w/w of yellow (propylene glycol lecithin, yellow 5 lake).

In some embodiments, any of the lip scrub compositions disclosed herein comprise at least about 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, or 2% w/w of green (propylene glycol lecithin, yellow 5 lake, blue 1). In some embodiments, any of the lip scrub compositions disclosed herein comprise at least about 0.1% w/w of green (propylene glycol lecithin, yellow 5 lake, blue 1). In some embodiments, any of the lip scrub compositions disclosed herein comprise at least about 0.2% w/w of green (propylene glycol lecithin, yellow 5 lake, blue 1). In some embodiments, any of the lip scrub compositions disclosed herein comprise at least about 0.3% w/w of green (propylene glycol lecithin, yellow 5 lake, blue 1). In some embodiments, any of the lip scrub compositions disclosed herein comprise at least about 0.5% w/w of green (propylene glycol lecithin, yellow 5 lake, blue 1). In some embodiments, any of the lip scrub compositions disclosed herein comprise at least about 0.7% w/w of green (propylene glycol lecithin, yellow 5 lake, blue 1). In some embodiments, any of the lip scrub compositions disclosed herein comprise at least about 1% w/w of green (propylene glycol lecithin, yellow 5 lake, blue 1).

Vitamin

In some embodiments, any of the lip scrub compositions disclosed herein comprise a vitamin. In some embodiments, any of the lip scrub compositions disclosed herein comprise 1, 2, 3, 4 or 5 or more vitamins.

In some embodiments, the vitamin is selected from vitamin C, vitamin E, and vitamin K1. In some embodiments, any of the lip scrub compositions disclosed herein vitamin C. In some embodiments, any of the lip scrub compositions disclosed herein E. In some embodiments, any of the lip scrub compositions disclosed herein vitamin K1. In some embodiments, any of the lip scrub compositions disclosed herein do not comprise vitamin K1.

In some embodiments, any of the lip scrub compositions disclosed herein comprise about 0% to about 10% w/w of the vitamin(s). In some embodiments, any of the lip scrub compositions disclosed herein comprise about 0.1% to about 10% w/w of the vitamin(s). In some embodiments, any of the lip scrub compositions disclosed herein comprise about 0.2% to about 10% w/w of the vitamin(s). In some embodiments, any of the lip scrub compositions disclosed herein comprise about 0.5% to about 10% w/w of the vitamin(s). In some embodiments, any of the lip scrub compositions disclosed herein comprise about 0% to about 7% w/w of the vitamin(s). In some embodiments, any of the lip scrub compositions disclosed herein comprise about 0% to about 5% w/w of the vitamin(s). In some embodiments, any of the lip scrub compositions disclosed herein comprise about 0% to about 3% w/w of the vitamin(s). In some embodiments, any of the lip scrub compositions disclosed herein comprise about 0% to about 2% w/w of the vitamin(s). In some embodiments, any of the lip scrub compositions disclosed herein comprise about 0% to about 1% w/w of the vitamin(s). In some embodiments, any of the lip scrub compositions disclosed herein comprise about 0.1% to about 7% w/w of the vitamin(s). In some embodiments, any of the lip scrub compositions disclosed herein comprise about 0.1% to about 5% w/w of the vitamin(s). In some embodiments, any of the lip scrub compositions disclosed herein comprise about 0.1% to about 3% w/w of the vitamin(s). In some embodiments, any of the lip scrub compositions disclosed herein comprise about 0.1% to about 2% w/w of the vitamin(s). In some embodiments, any of the lip scrub compositions disclosed herein comprise about 0.1% to about 1% w/w of the vitamin(s). In some embodiments, any of the lip scrub compositions disclosed herein comprise about 0.3% to about 7% w/w of the vitamin(s). In some embodiments, any of the lip scrub compositions disclosed herein comprise about 0.3% to about 5% w/w of the vitamin(s). In some embodiments, any of the lip scrub compositions disclosed herein comprise about 0.3% to about 3% w/w of the vitamin(s). In some embodiments, any of the lip scrub compositions disclosed herein comprise about 0.3% to about 2% w/w of the vitamin(s). In some embodiments, any of the lip scrub compositions disclosed herein comprise about 0.3% to about 1% w/w of the vitamin(s). In some embodiments, any of the lip scrub compositions disclosed herein comprise about 0.5% to about 7% w/w of the vitamin(s). In some embodiments, any of the lip scrub compositions disclosed herein comprise about 0.5% to about 5% w/w of the vitamin(s). In some embodiments, any of the lip scrub compositions disclosed herein comprise about 0.5% to about 3% w/w of the vitamin(s). In some embodiments, any of the lip scrub compositions disclosed herein comprise about 0.5% to about 2% w/w of the vitamin(s). In some embodiments, any of the lip scrub compositions disclosed herein comprise about 0.5% to about 1% w/w of the vitamin(s). In some embodiments, any of the lip scrub compositions disclosed herein comprise about 0% to about 1% w/w of the vitamin(s).

In some embodiments, any of the lip scrub compositions disclosed herein comprise at least about 0.1%, 0.2%, 0.3%, 0.4%, 0.5%, 0.6%, 0.7%, 0.8%, 0.9%, 1%, 1.1%, 1.2%, 1.3%, 1.4%, 1.5%, 1.6%, 1.7%, 1.8%, 1.9%, or 2% w/w of the vitamin(s). In some embodiments, any of the lip scrub compositions disclosed herein comprise at least about 0.1% w/w of the vitamin(s). In some embodiments, any of the lip scrub compositions disclosed herein comprise at least about 0.2% w/w of the vitamin(s). In some embodiments, any of the lip scrub compositions disclosed herein comprise at least about 0.3% w/w of the vitamin(s). In some embodiments, any of the lip scrub compositions disclosed herein comprise at least about 0.5% w/w of the vitamin(s). In some embodiments, any of the lip scrub compositions disclosed herein comprise at least about 0.7% w/w of the vitamin(s). In some embodiments, any of the lip scrub compositions disclosed herein comprise at least about 1% w/w of the vitamin(s).

Humectant

In some embodiments, any of the lip scrub compositions disclosed herein comprise a humectant. In some embodiments, any of the lip scrub compositions disclosed herein comprise 1, 2, 3, 4, or 5 or more humectants. In some embodiments, any of the lip scrub compositions disclosed herein comprise 2 or more humectants. In some embodiments, any of the lip scrub compositions disclosed herein comprise 3 or more humectants.

In some embodiments, the humectant is selected from: Crambe abyssinica seed oil, glycerin, lecithin, and propylene glycol.

In some embodiments, any of the lip scrub compositions disclosed herein comprise about 0% to about 15% w/w of the humectant(s). In some embodiments, any of the lip scrub compositions disclosed herein comprise about 1% to about 30% w/w of the humectant(s). In some embodiments, any of the lip scrub compositions disclosed herein comprise about 1% to about 25% w/w of the humectant(s). In some embodiments, any of the lip scrub compositions disclosed herein comprise about 5% to about 20% w/w of the humectant(s). In some embodiments, any of the lip scrub compositions disclosed herein comprise about 1% to about 15% w/w of the humectant(s). In some embodiments, any of the lip scrub compositions disclosed herein comprise about 5% to about 30% w/w of the humectant(s). In some embodiments, any of the lip scrub compositions disclosed herein comprise about 5% to about 25% w/w of the humectant(s). In some embodiments, any of the lip scrub compositions disclosed herein comprise about 5% to about 20% w/w of the humectant(s). In some embodiments, any of the lip scrub compositions disclosed herein comprise about 5% to about 15% w/w of the humectant(s). In some embodiments, any of the lip scrub compositions disclosed herein comprise about 5% to about 10% w/w of the humectant(s). In some embodiments, any of the lip scrub compositions disclosed herein comprise about 0% to about 10% w/w of the humectant(s).

In some embodiments, any of the lip scrub compositions disclosed herein comprise at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, or 15% w/w of the humectant(s). In some embodiments, any of the lip scrub compositions disclosed herein comprise at least about 1% w/w of the humectant(s). In some embodiments, any of the lip scrub compositions disclosed herein comprise at least about 5% w/w of the humectant(s). In some embodiments, any of the lip scrub compositions disclosed herein comprise at least about 10% w/w of the humectant(s). In some embodiments, any of the lip scrub compositions disclosed herein comprise at least about 15% w/w of the humectant(s).

In some embodiments, the humectant is propylene glycol In some embodiments, any of the lip scrub compositions disclosed herein comprise at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, or 15% w/w of the polypropylene glycol. In some embodiments, any of the lip scrub compositions disclosed herein comprise at least about 1% w/w of the polypropylene glycol. In some embodiments, any of the lip scrub compositions disclosed herein comprise at least about 5% w/w of the polypropylene glycol. In some embodiments, any of the lip scrub compositions disclosed herein comprise at least about 10% w/w of the polypropylene glycol. In some embodiments, any of the lip scrub compositions disclosed herein comprise at least about 15% w/w of the polypropylene glycol.

Preservative

In some embodiments, any of the lip scrub compositions disclosed herein comprise a preservative. In some embodiments, any of the lip scrub compositions disclosed herein comprise 1, 2, 3, 4, or 5 or more preservatives.

In some embodiments, any of the lip scrub compositions disclosed herein comprise a preservative. In some embodiments, the preservative is selected from: benyzl alcohol, dehydroacetic acid, glyceryl caprilate, hydroxyacetophenone, potassium sorbate, caprylhydroxamic acid, caprylyl glycol, lycerin, geogard ECT, sensicare M4200, Geogard Ultra, anisic acid, glyceryl caprylate, glyceryl undecylenate, naticide, ehtyl lauroyl arginate, and biosecur.

In some embodiments, any of the lip scrub compositions disclosed herein comprise about 0% to 5% w/w of the preservative(s). In some embodiments, any of the lip scrub compositions disclosed herein comprise about 0% to 4% w/w of the preservative(s). In some embodiments, any of the lip scrub compositions disclosed herein comprise about 0% to 3% w/w of the preservative(s). In some embodiments, any of the lip scrub compositions disclosed herein comprise about 0% to 2% w/w of the preservative(s). In some embodiments, any of the lip scrub compositions disclosed herein comprise about 0% to 1% w/w of the preservative(s). In some embodiments, any of the lip scrub compositions disclosed herein comprise about 0% to 0.5% w/w of the preservative(s). In some embodiments, any of the lip scrub compositions disclosed herein comprise about 0.1% to 3% w/w of the preservative(s). In some embodiments, any of the lip scrub compositions disclosed herein comprise about 0.1% to 2% w/w of the preservative(s).

In some embodiments, any of the lip scrub compositions disclosed herein comprise about 0.1% to 1% w/w of the preservative(s). In some embodiments, any of the lip scrub compositions disclosed herein comprise about 0.15% to 3% w/w of the preservative(s). In some embodiments, any of the lip scrub compositions disclosed herein comprise about 0.15% to 2% w/w of the preservative(s). In some embodiments, any of the lip scrub compositions disclosed herein comprise about 0.15% to 1% w/w of the preservative(s). In some embodiments, any of the lip scrub compositions disclosed herein comprise about 0.15% to 0.5% w/w of the preservative(s). In some embodiments, any of the lip scrub compositions disclosed herein comprise about 0.15% to 0.3% w/w of the preservative(s).

In some embodiments, any of the lip scrub compositions disclosed herein comprise at least about 0.01%, 0.05%, 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, or 0.45% or more w/w of the preservative(s). In some embodiments, any of the lip scrub compositions disclosed herein comprise at least about 0.01% or more w/w of the preservative(s). In some embodiments, any of the lip scrub compositions disclosed herein comprise at least about 0.05% or more w/w of the preservative(s). In some embodiments, any of the lip scrub compositions disclosed herein comprise at least about 0.15% or more w/w of the preservative(s). In some embodiments, any of the lip scrub compositions disclosed herein comprise at least about 0.2% or more w/w of the preservative(s). In some embodiments, any of the lip scrub compositions disclosed herein comprise at least about 0.25% or more w/w of the preservative(s). In some embodiments, any of the lip scrub compositions disclosed herein comprise at least about 0.3% or more w/w of the preservative(s).

In some embodiments, the preservative is potassium sorbate. In some embodiments, any of the lip scrub compositions disclosed herein comprise at least about 0.01%, 0.05%, 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, or 0.45% or more w/w of potassium sorbate. In some embodiments, any of the lip scrub compositions disclosed herein comprise at least about 0.01% or more w/w of potassium sorbate. In some embodiments, any of the lip scrub compositions disclosed herein comprise at least about 0.05% or more w/w of potassium sorbate. In some embodiments, any of the lip scrub compositions disclosed herein comprise at least about 0.15% or more w/w of potassium sorbate. In some embodiments, any of the lip scrub compositions disclosed herein comprise at least about 0.2% or more w/w of potassium sorbate. In some embodiments, any of the lip scrub compositions disclosed herein comprise at least about 0.25% or more w/w of potassium sorbate. In some embodiments, any of the lip scrub compositions disclosed herein comprise at least about 0.3% or more w/w of potassium sorbate.

Abrasive

In some embodiments, any of the lip scrub compositions disclosed herein comprise an abrasive. In some embodiments, any of the lip scrub compositions disclosed herein comprise 1, 2, 3, 4, or 5 or more abrasives. In some embodiments, any of the lip scrub compositions disclosed herein comprise 2 or more abrasives. In some embodiments, any of the lip scrub compositions disclosed herein comprise 3 or more abrasives.

In some embodiments, the abrasive is not a sugar alcohol. In some embodiments, the abrasive is not a sugar.

In some embodiments, abrasive is selected from sugar-free sprinkles nonpareil, fruit peel, fruit powder, vegetable peel, vegetable powder, and jojoba beads. In some embodiments, the fruit peel is selected from orange peel, grapefruit peel, carrot peel, pumpkin peel, and lemon peel. In some embodiments, the fruit peel is carrot peel. In some embodiments, the fruit peel is pumpkin peel. In some embodiments, the abrasive is in the form of a granule.

In some embodiments, any of the lip scrub compositions disclosed herein comprise about 0% to about 15% w/w of the abrasive(s). In some embodiments, any of the lip scrub compositions disclosed herein comprise about 1% to about 30% w/w of the abrasive(s). In some embodiments, any of the lip scrub compositions disclosed herein comprise about 1% to about 25% w/w of the abrasive(s). In some embodiments, any of the lip scrub compositions disclosed herein comprise about 5% to about 20% w/w of the abrasive(s). In some embodiments, any of the lip scrub compositions disclosed herein comprise about 1% to about 15% w/w of the abrasive(s). In some embodiments, any of the lip scrub compositions disclosed herein comprise about 5% to about 30% w/w of the abrasive(s). In some embodiments, any of the lip scrub compositions disclosed herein comprise about 5% to about 25% w/w of the abrasive(s). In some embodiments, any of the lip scrub compositions disclosed herein comprise about 5% to about 20% w/w of the abrasive(s). In some embodiments, any of the lip scrub compositions disclosed herein comprise about 5% to about 15% w/w of the abrasive(s). In some embodiments, any of the lip scrub compositions disclosed herein comprise about 5% to about 10% w/w of the abrasive(s). In some embodiments, any of the lip scrub compositions disclosed herein comprise about 0% to about 10% w/w of the abrasive(s).

In some embodiments, any of the lip scrub compositions disclosed herein comprise at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, or 15% w/w of the abrasive(s). In some embodiments, any of the lip scrub compositions disclosed herein comprise at least about 1% w/w of the abrasive(s). In some embodiments, any of the lip scrub compositions disclosed herein comprise at least about 5% w/w of the abrasive(s). In some embodiments, any of the lip scrub compositions disclosed herein comprise at least about 10% w/w of the abrasive(s). In some embodiments, any of the lip scrub compositions disclosed herein comprise at least about 15% w/w of the abrasive(s).

In some embodiments, any of the lip scrub compositions disclosed herein comprise at least about 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, or 15% w/w of orange peel granules. In some embodiments, any of the lip scrub compositions disclosed herein comprise at least about 1% w/w of orange peel granules. In some embodiments, any of the lip scrub compositions disclosed herein comprise at least about 5% w/w of orange peel granules. In some embodiments, any of the lip scrub compositions disclosed herein comprise at least about 10% w/w of orange peel granules. In some embodiments, any of the lip scrub compositions disclosed herein comprise at least about 15% w/w of orange peel granules.

Water

In some embodiments, any of the lip scrub compositions disclosed herein comprise water. In some embodiments, any of the lip scrub compositions disclosed herein comprise less than about 95%, 90%, 80%, 70%, 60%, 50%, 40%, 30%, 20%, 10%, 9%, 8%, 7%, 6%, 5%, 4%, 3%, 2%, or 1% w/v water. In some embodiments, any of the lip scrub compositions disclosed herein comprise less than about 50% w/v water. In some embodiments, any of the lip scrub compositions disclosed herein comprise less than about 30% w/v water. In some embodiments, any of the lip scrub compositions disclosed herein comprise less than about 20% w/v water. In some embodiments, any of the lip scrub compositions disclosed herein comprise less than about 10% w/v water. In some embodiments, any of the lip scrub compositions disclosed herein comprise less than about 5% w/v water. In some embodiments, any of the lip scrub compositions disclosed herein comprise less than about 1% w/v water.

Lip Plumping Agent

In some embodiments, any of the lip scrub compositions disclosed herein comprise a lip plumping agent. In some embodiments, any of the lip scrub compositions disclosed herein comprise 1, 2, 3, 4, or 5 or more lip plumping agents. In some embodiments, any of the lip scrub compositions disclosed herein comprise 2 or more lip plumping agents. In some embodiments, any of the lip scrub compositions disclosed herein comprise 3 or more lip plumping agents.

In some embodiments, the lip plumping agent is selected from sodium hyaluronate, cinnamon, capsicum, and menthol. In some embodiments, the lip plumping agent is sodium hyaluronate.

In some embodiments, any of the lip scrub compositions disclosed herein comprise about 0% to about 7% w/w of the lip plumping agent(s). In some embodiments, any of the lip scrub compositions disclosed herein comprise about 0% to about 5% w/w of the lip plumping agent(s). In some embodiments, any of the lip scrub compositions disclosed herein comprise about 0% to about 4% w/w of the lip plumping agent(s). In some embodiments, any of the lip scrub compositions disclosed herein comprise about 0% to about 3% w/w of the lip plumping agent(s). In some embodiments, any of the lip scrub compositions disclosed herein comprise about 0.01% to about 5% w/w of the lip plumping agent(s). In some embodiments, any of the lip scrub compositions disclosed herein comprise about 0.01% to about 4% w/w of the lip plumping agent(s). In some embodiments, any of the lip scrub compositions disclosed herein comprise about 0.01% to about 3% w/w of the lip plumping agent(s). In some embodiments, any of the lip scrub compositions disclosed herein comprise about 0.1% to about 5% w/w of the lip plumping agent(s). In some embodiments, any of the lip scrub compositions disclosed herein comprise about 0.1% to about 4% w/w of the lip plumping agent(s). In some embodiments, any of the lip scrub compositions disclosed herein comprise about 0.1% to about 3% w/w of the lip plumping agent(s).

In some embodiments, any of the lip scrub compositions disclosed herein comprise at least about 0.001%, 0.002%, 0.003%, 0.004%, 0.005%, 0.01%, 0.011%, 0.012%, 0.013%, 0.014%, 0.015%, 0.016%, 0.017%, 0.018%, 0.019%, 0.02%, 0.025%, 0.03%, 0.035%, 0.04%, 0.045%, 0.05%, 0.055%, 0.06%, 0.065%, 0.07%, 0.075%, 0.08%, 0.085%, 0.09%, 0.095%, 0.1%, 0.11%, 0.12%, 0.13%, 0.14%, 0.15%, 0.16%, 0.17%, 0.18%, 0.19%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.1%, 1.11%, 1.12%, 1.13%, 1.14%, 1.15%, 1.16%, 1.17%, 1.18%, 1.19%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, 2%, 2.1%, 2.11%, 2.12%, 2.13%, 2.14%, 2.15%, 2.16%, 2.17%, 2.18%, 2.19%, 2.2%, 2.25%, 2.3%, 2.35%, 2.4%, 2.45%, 2.5%, 2.55%, 2.6%, 2.65%, 2.7%, 2.75%, 2.8%, 2.85%, 2.9%, 2.95%, 3%, 3.1%, 3.11%, 3.12%, 3.13%, 3.14%, 3.15%, 3.16%, 3.17%, 3.18%, 3.19%, 3.2%, 3.25%, 3.3%, 3.35%, 3.4%, 3.45%, 3.5%, 3.55%, 3.6%, 3.65%, 3.7%, 3.75%, 3.8%, 3.85%, 3.9%, 3.95%, or 4% w/w of the lip plumping agent(s). In some embodiments, any of the lip scrub compositions disclosed herein comprise at least about 0.001% w/w of the lip plumping agent(s). In some embodiments, any of the lip scrub compositions disclosed herein comprise at least about 0.01% w/w of the lip plumping agent(s). In some embodiments, any of the lip scrub compositions disclosed herein comprise at least about 0.05% w/w of the lip plumping agent(s). In some embodiments, any of the lip scrub compositions disclosed herein comprise at least about 0.1% w/w of the lip plumping agent(s). In some embodiments, any of the lip scrub compositions disclosed herein comprise at least about 0.15% w/w of the lip plumping agent(s).

Drug Agent

In some embodiments, any of the lip scrub compositions disclosed herein comprise a drug agent. In some embodiments, any of the lip scrub compositions disclosed herein comprise 1, 2, 3, 4, or 5 or more drug agents. In some embodiments, the drug agent is an agent that can be applied topically. In some embodiments, the drug agent is an agent that can be absorbed through the skin.

In some embodiments, the drug agent is selected from a calming agent, a keratolytic agent, and caffeine. In some embodiments, a keratolytic agent is an agent capable of softening and separating the cornified (e.g., tough) layer of skin. In some embodiments, the keratolytic agent is an agent that facilitates or promotes shedding of dead skin cells. In some embodiments, the keratolytic agent is allantoin. In some embodiments, the calming agent is cannabidol (cbd) oil.

In some embodiments, any of the lip scrub compositions disclosed herein comprise about 0% to about 7% w/w of the drug agent(s). In some embodiments, any of the lip scrub compositions disclosed herein comprise about 0% to about 5% w/w of the drug agent(s). In some embodiments, any of the lip scrub compositions disclosed herein comprise about 0% to about 4% w/w of the drug agent(s). In some embodiments, any of the lip scrub compositions disclosed herein comprise about 0% to about 3% w/w of the drug agent(s). In some embodiments, any of the lip scrub compositions disclosed herein comprise about 0.01% to about 5% w/w of the drug agent(s). In some embodiments, any of the lip scrub compositions disclosed herein comprise about 0.01% to about 4% w/w of the drug agent(s). In some embodiments, any of the lip scrub compositions disclosed herein comprise about 0.01% to about 3% w/w of the drug agent(s). In some embodiments, any of the lip scrub compositions disclosed herein comprise about 0.1% to about 5% w/w of the drug agent(s). In some embodiments, any of the lip scrub compositions disclosed herein comprise about 0.1% to about 4% w/w of the drug agent(s). In some embodiments, any of the lip scrub compositions disclosed herein comprise about 0.1% to about 3% w/w of the drug agent(s).

In some embodiments, any of the lip scrub compositions disclosed herein comprise at least about 0.001%, 0.002%, 0.003%, 0.004%, 0.005%, 0.01%, 0.011%, 0.012%, 0.013%, 0.014%, 0.015%, 0.016%, 0.017%, 0.018%, 0.019%, 0.02%, 0.025%, 0.03%, 0.035%, 0.04%, 0.045%, 0.05%, 0.055%, 0.06%, 0.065%, 0.07%, 0.075%, 0.08%, 0.085%, 0.09%, 0.095%, 0.1%, 0.11%, 0.12%, 0.13%, 0.14%, 0.15%, 0.16%, 0.17%, 0.18%, 0.19%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.1%, 1.11%, 1.12%, 1.13%, 1.14%, 1.15%, 1.16%, 1.17%, 1.18%, 1.19%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, 2%, 2.1%, 2.11%, 2.12%, 2.13%, 2.14%, 2.15%, 2.16%, 2.17%, 2.18%, 2.19%, 2.2%, 2.25%, 2.3%, 2.35%, 2.4%, 2.45%, 2.5%, 2.55%, 2.6%, 2.65%, 2.7%, 2.75%, 2.8%, 2.85%, 2.9%, 2.95%, 3%, 3.1%, 3.11%, 3.12%, 3.13%, 3.14%, 3.15%, 3.16%, 3.17%, 3.18%, 3.19%, 3.2%, 3.25%, 3.3%, 3.35%, 3.4%, 3.45%, 3.5%, 3.55%, 3.6%, 3.65%, 3.7%, 3.75%, 3.8%, 3.85%, 3.9%, 3.95%, or 4% w/w of the drug agent(s). In some embodiments, any of the lip scrub compositions disclosed herein comprise at least about 0.001% w/w of the drug agent(s). In some embodiments, any of the lip scrub compositions disclosed herein comprise at least about 0.01% w/w of the drug agent(s). In some embodiments, any of the lip scrub compositions disclosed herein comprise at least about 0.05% w/w of the drug agent(s). In some embodiments, any of the lip scrub compositions disclosed herein comprise at least about 0.1% w/w of the drug agent(s). In some embodiments, any of the lip scrub compositions disclosed herein comprise at least about 0.15% w/w of the drug agent(s).

In some embodiments, any of the lip scrub compositions disclosed herein comprise at least about 0.001%, 0.002%, 0.003%, 0.004%, 0.005%, 0.01%, 0.011%, 0.012%, 0.013%, 0.014%, 0.015%, 0.016%, 0.017%, 0.018%, 0.019%, 0.02%, 0.025%, 0.03%, 0.035%, 0.04%, 0.045%, 0.05%, 0.055%, 0.06%, 0.065%, 0.07%, 0.075%, 0.08%, 0.085%, 0.09%, 0.095%, 0.1%, 0.11%, 0.12%, 0.13%, 0.14%, 0.15%, 0.16%, 0.17%, 0.18%, 0.19%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1%, 1.1%, 1.11%, 1.12%, 1.13%, 1.14%, 1.15%, 1.16%, 1.17%, 1.18%, 1.19%, 1.2%, 1.25%, 1.3%, 1.35%, 1.4%, 1.45%, 1.5%, 1.55%, 1.6%, 1.65%, 1.7%, 1.75%, 1.8%, 1.85%, 1.9%, 1.95%, 2%, 2.1%, 2.11%, 2.12%, 2.13%, 2.14%, 2.15%, 2.16%, 2.17%, 2.18%, 2.19%, 2.2%, 2.25%, 2.3%, 2.35%, 2.4%, 2.45%, 2.5%, 2.55%, 2.6%, 2.65%, 2.7%, 2.75%, 2.8%, 2.85%, 2.9%, 2.95%, 3%, 3.1%, 3.11%, 3.12%, 3.13%, 3.14%, 3.15%, 3.16%, 3.17%, 3.18%, 3.19%, 3.2%, 3.25%, 3.3%, 3.35%, 3.4%, 3.45%, 3.5%, 3.55%, 3.6%, 3.65%, 3.7%, 3.75%, 3.8%, 3.85%, 3.9%, 3.95%, or 4% w/w of cbd oil. In some embodiments, any of the lip scrub compositions disclosed herein comprise at least about 0.001% w/w of cbd oil. In some embodiments, any of the lip scrub compositions disclosed herein comprise at least about 0.01% w/w of cbd oil. In some embodiments, any of the lip scrub compositions disclosed herein comprise at least about 0.05% w/w of cbd oil. In some embodiments, any of the lip scrub compositions disclosed herein comprise at least about 0.1% w/w of cbd oil. In some embodiments, any of the lip scrub compositions disclosed herein comprise at least about 0.15% w/w of cbd oil.

Formulation

In some embodiments, the lip scrub composition is formulated as a lip balm, lip stick, lip cream, lip gel, or lip gloss. In some embodiments, the lip scrub composition is translucent. In some embodiments, a translucent lip scrub composition is free of added color or tint, such that when the translucent lip scrub composition is applied to the lips, it does not add color or tint to the lips. In some embodiments, the lip scrub composition is tinted. In some embodiments, a tinted lip scrub composition contains a colorant or tint that adds color or tint to the lips once the tinted lip scrub composition is applied to the lips.

In some embodiments, any of the lip scrub compositions disclosed herein have a shelf life of at least 6, 7, 8, 9, 10, 11, 12, 13, 14, 15, 16, 17, 18, 19, 20, 21, 22, 23, or 24 months when stored in a cool place, out of direct sunlight. In some embodiments, any of the lip scrub compositions disclosed herein have a shelf life of at least 6 months when stored in a cool place, out of direct sunlight. In some embodiments, any of the lip scrub compositions disclosed herein have a shelf life of at least 12 months when stored in a cool place, out of direct sunlight. In some embodiments, any of the lip scrub compositions disclosed herein have a shelf life of at least 18 months when stored in a cool place, out of direct sunlight. In some embodiments, any of the lip scrub compositions disclosed herein have a shelf life of at least 24 months when stored in a cool place, out of direct sunlight. In some embodiments, a cool place is a place having a temperature of less than about 75°, 70°, 69°, 68°, 67°, 66°, 65°, 64°, 63°, 62°, 61° or 60° C. In some embodiments, a cool place is a place having a temperature of less than about 75° C. In some embodiments, a cool place is a place having a temperature of less than about 70° C. In some embodiments, a cool place is a place having a temperature of less than about 65° C. In some embodiments, a cool place is a place having a temperature of less than about 60° C. In some embodiments, a cool place is a place having a temperature of less than about 55° C. In some embodiments, any of the lip scrub compositions disclosed herein have a shelf life of at least 12 months when stored at less than about 75° C., out of direct sunlight. In some embodiments, any of the lip scrub compositions disclosed herein have a shelf life of at least 12 months when stored at less than about 70° C., out of direct sunlight. In some embodiments, any of the lip scrub compositions disclosed herein have a shelf life of at least 12 months when stored at less than about 65° C., out of direct sunlight.

Lip Scrub Product

Further disclosed herein is a lip scrub product comprising any of the lip scrub compositions disclosed herein. In some embodiments, the lip scrub composition is contained in a container of any shape or size. In some embodiments, the container can be made of any material, such as glass or plastic. The container may be clear. The container may be colorless. The container may be opaque. In some embodiments, the container is a jar or tube.

In some embodiments, the lip scrub product further comprises an applicator. In some embodiments, the applicator is used to apply the lip scrub composition to the lips.

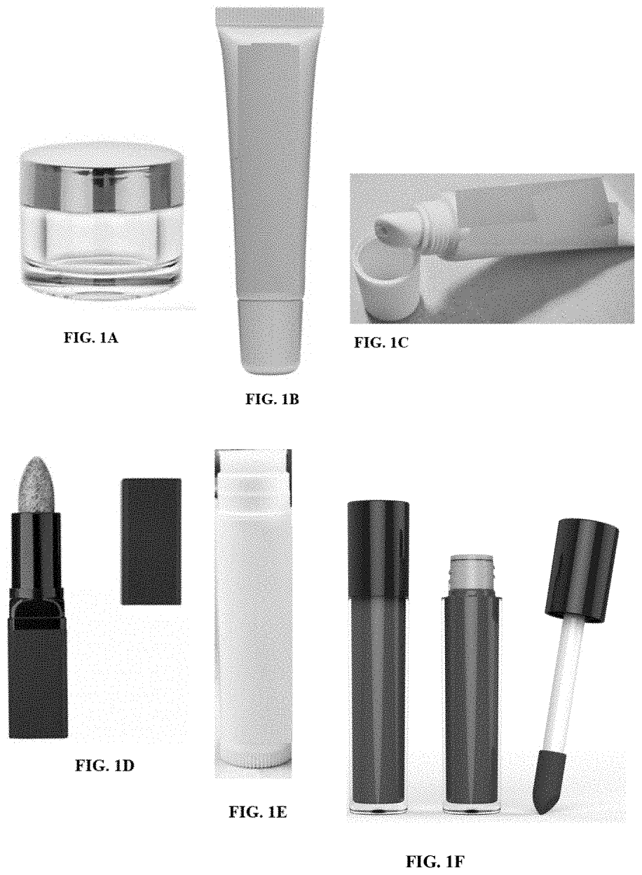

Exemplary containers are shown in FIGS. 1A-1F. As shown in FIG. 1A, any of the lip scrub compositions disclosed herein may be contained in a container that is a jar. In some embodiments, the jar comprises a top portion, which is a cap, and a bottom portion that holds the lip scrub composition.

As shown in FIGS. 1B and 1C, any of the lip scrub compositions disclosed herein may be contained in a container that is a tube. The tub may comprise a first portion that holds the lip scrub composition and a second portion that is a cap. The first portion may comprise a hole that allows the lip scrub composition to be released from the tube.

As shown in FIG. 1D, any of the lip scrub compositions disclosed herein may be in the form of a lip stick. The lip stick may comprise a first portion that holds the lip scrub composition and a second portion that is a cap. The first portion may further comprise a mechanism for raising and lowering the lip scrub composition in the first portion.

As shown in FIG. 1E, any of the lip scrub compositions disclosed herein may be in the form of a lip balm. The lip balm may comprise a first portion that holds the lip scrub composition and a second portion that is a cap. The first portion may further comprise a mechanism for raising and lowering the lip scrub composition in the first portion.

As shown in FIG. 1F, any of the lip scrub compositions disclosed herein may be in the form of a lip gloss. The lip gloss may comprise a first portion that holds the lip scrub composition and a second portion that is a cap. The second portion may further comprise an applicator that is used to apply the lip scrub composition to the lips.

Methods

Further disclosed herein is a method of exfoliating lips of a subject, the method comprising topically administering to the lips of the subject any of the lip scrub compositions disclosed herein. In some embodiments, the lip scrub composition is applied directly to the lips. In some embodiments, the lip scrub composition is applied to the lips via an applicator. In some embodiments, the lip scrub composition is rubbed onto the subject's fingers and then applied to the lips.

Further disclosed herein is a method of exfoliating lips of a subject, the method comprising topically administering to the lips of the subject any of the lip scrub products disclosed herein. In some embodiments, the lip scrub composition is applied directly to the lips. In some embodiments, the lip scrub composition is applied to the lips via an applicator. In some embodiments, the lip scrub composition is rubbed onto the subject's fingers and then applied to the lips.

### Definitions

Unless defined otherwise, all technical and scientific terms used herein have the meaning commonly understood by a person skilled in the art to which this disclosure belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al., (eds.), Springer Verlag (1991); and Hale & Marham, The Harper Collins Dictionary of Biology (1991). As used herein, the following terms have the meanings ascribed to them below, unless specified otherwise. The terminology used herein is for the purpose of describing particular embodiments only and is not intended to be limiting of the disclosure.

The terms “a” and “an” as used herein mean “one or more” and include the plural unless the context is inappropriate.

The term “about” as used herein when referring to a measurable value (e.g., weight, time, and dose) is meant to encompass variations, such as ±10%, ±5% , ±1% , or ±0.1% of the specified value.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

As a general matter, compositions specifying a percentage are by weight unless otherwise specified. Further, if a variable is not accompanied by a definition, then the previous definition of the variable controls.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates that may need to be independently confirmed.

### EXAMPLES

### Example 1. Sugar-Free Lip Scrub Composition

This example describes an exemplary sugar-free lip scrub composition. As shown below, a sugar-free lip scrub composition may comprise (a) an exfoliating agent, such as a sugar alcohol; (b) one or more emollients; (c) a flavoring agent; (d) one or more colorants; and (e) a preservative.

| Percent (w/w) | Ingredient | Description |
|---|---|---|
| 80%-95% | erythritol (sugar alcohol) | exfoliating agent |
| 5%-15% | pumpkin seed oil | emollient |
| 5%-15% | grapeseed oil | emollient |
| 1%-3% | natural orange oil | flavoring agent |
| 0.5%-1% | orange (propylene glycol lecithin, | colorant |
| | yellow 6 lake) | |
| 0.5%-1% | yellow (propylene glycol lecithin, | colorant |
| | yellow 5 lake) | |
| .15%-.3% | potassium sorbate | preservative |

### Example 2. Sugar-Free Lip Scrub Composition

This example describes an exemplary sugar-free lip scrub composition. As shown below, a sugar-free lip scrub composition may comprise (a) an exfoliating agent, such as a sugar alcohol; (b) one or more emollients; (c) one or more flavoring agents; (d) one or more colorants; (e) a lip plumping agent and/or moisturizer; and (f) a preservative.

| Percent (w/w) | Ingredient | Description |
|---|---|---|
| 80%-95% | erythritol (sugar alcohol) | exfoliating agent |
| 5%-15% | pumpkin seed oil | emollient |
| 5%-15% | grapeseed oil | emollient |
| 5%-10% | orange peel granules | abrasive |
| 1%-3% | natural orange oil | flavoring agent |
| 1%-3% | limonene (terpene extract) | flavoring agent |
| 0.5%-1% | orange (propylene glycol lecithin, | colorant |
| | yellow 6 lake) | |
| 0.5%-1% | yellow (propylene glycol lecithin, | colorant |
| | yellow 5 lake) | |
| 0.5%-1% | stevia extract | flavoring agent |
| 0.1%-0.5% | hyaluronic acid | lip plumping agent |
| | | and/or moisturizer |
| 0.15%-0.3% | potassium sorbate | preservative |

### Example 3. Sugar-Free Lip Scrub Composition

This example describes an exemplary sugar-free lip scrub composition. As shown below, a sugar-free lip scrub composition may comprise (a) an exfoliating agent, such as a sugar alcohol; (b) an emollient; (c) an abrasive; (d) one or more flavoring agents; (e) a colorant; (f) a drug; and (g) a preservative.

| Percent (w/w) | Ingredient | Description |
|---|---|---|
| 80%-95% | erythritol (sugar alcohol) | exfoliating agent |
| 5%-15% | hempseed oil | emollient |
| 5%-10% | orange peel granules | abrasive |
| 1%-3% | terpene extracts | flavoring agent |
| 0.5%-1% | green (propolyne glycol | colorant |
| | lecithin, yellow 5 lake, blue 1) | |
| 0.5%-1% | stevia extract | flavoring agent |
| 0.1%-1.5% | cbd oil (cannabidiol) | drug, calming agent |
| 0.15%-0.3% | potassium sorbate | preservative |

### Example 4. Sugar-Free Lip Scrub Composition

This example describes an exemplary sugar-free lip scrub composition. As shown below, a sugar-free lip scrub composition may comprise (a) an exfoliating agent, such as a sugar alcohol; (b) one or more emollients; (c) a flavoring agent; (d) one or more colorants; and (e) a preservative.

| Percent (w/w) | Ingredient | Description |
|---|---|---|
| 80%-95% | erythritol (sugar alcohol) | exfoliating agent |
| 5%-15% | Camelina oil | emollient |
| 5%-15% | cranberry seed oil | emollient |
| 1%-3% | natural orange oil | flavoring agent |
| 0.5%-1% | orange (propylene glycol lecithin, | colorant |
| | yellow 6 lake) | |
| 0.5%-1% | yellow (propylene glycol lecithin, | colorant |
| | yellow 5 lake) | |
| .15%-.3% | potassium sorbate | preservative |

### Example 5. Sugar-Free Lip Scrub Composition

This example describes an exemplary sugar-free lip scrub composition. As shown below, a sugar-free lip scrub composition may comprise (a) an exfoliating agent, such as a sugar alcohol; (b) one or more emollients; (c) one or more flavoring agents; (d) one or more colorants; (e) a lip plumping agent and/or moisturizer; and (f) a preservative.

| Percent (w/w) | Ingredient | Description |
|---|---|---|
| 80%-95% | erythritol (sugar alcohol) | exfoliating agent |
| 5%-15% | Camelina oil | emollient |
| 5%-15% | cranberry seed oil | emollient |
| 5%-10% | orange peel granules | abrasive |
| 1%-3% | natural orange oil | flavoring agent |
| 1%-3% | limonene (terpene extract) | flavoring agent |
| 0.5%-1% | orange (propylene glycol lecithin, | colorant |
| | yellow 6 lake) | |
| 0.5%-1% | yellow (propylene glycol lecithin, | colorant |
| | yellow 5 lake) | |
| 0.5%-1% | stevia extract | flavoring agent |
| 0.1%-0.5% | hyaluronic acid | lip plumping agent |
| | | and/or moisturizer |
| 0.15%-0.3% | potassium sorbate | preservative |

### Example 6. Sugar-Free Lip Scrub Composition

This example describes an exemplary sugar-free lip scrub composition. As shown below, a sugar-free lip scrub composition may comprise (a) an exfoliating agent, such as a sugar alcohol; (b) an emollient; (c) an abrasive; (d) one or more flavoring agents; (e) a colorant; (f) a drug; and (g) a preservative.

| Percent (w/w) | Ingredient | Description |
|---|---|---|
| 80%-95% | erythritol (sugar alcohol) | exfoliating agent |
| 5%-15% | hempseed oil | emollient |
| 5%-10% | orange peel granules | abrasive |
| 1%-3% | terpene extracts | flavoring agent |
| 0.5%-1% | green (propolyne glycol | colorant |
| | lecithin, yellow 5 lake, blue 1) | |
| 0.5%-1% | stevia extract | flavoring agent |
| 0.1%-1.5% | cbd oil (cannabidiol) | drug, calming agent |
| 0.15%-0.3% | potassium sorbate | preservative |

What is claimed is:

1. A lip scrub composition comprising an exfoliating agent that is a granulated sugar alcohol, wherein the lip scrub composition comprises less than about 80% w/w sugar.

2. The lip scrub composition of claim 1, wherein the sugar alcohol is selected from erythritol, xylitol, rnannitol, sorbitol, lactitol, isomalt, maltitol and hydrogenated starch hydrolysates (HSH).

3. The lip scrub composition of claim 1, wherein the lip scrub composition comprises about 75% to about 85% w/w of the exfoliating agent.

4. The lip scrub composition of claim 1, wherein the exfoliating agent is erythritol.

5. The lip scrub composition of claim 4, wherein the lip scrub composition comprises at least about 70% w/w of erythritol.

6. The lip scrub composition of claim 1, wherein the lip scrub composition further comprises an emollient, flavoring agent, thickening agent, essential oil, colorant, vitamin, humectant, preservative, or any combination thereof.

7. The lip scrub composition of claim 6, wherein the lip scrub composition comprises about 5% to about 15% w/w of the emollient, and wherein the emollient is selected from: Aloe vera oil, aprikot kernel oil, argan oil, avocado oil, babassu oil, blueberry seed oil, broccoli seed oil, Butyrospermum parkii unsaponifiables, Camellia kissi seed oil, Camelina oil, canola oil, carrot seed oil, castor oil, celery seed oil, coconut oil, Cocos nucifera (Coconut) Oil, cranberry seed oil, cucumber seed oil, dimethicone, flaxseed oil, glycerin, grapeseed oil, green tea oil, hazelnut oil, Helianthus annuus seed oil, hemp oil, hydrogenated moringa oil esters, jojoba oil, lanolin, lavender oil, macadamia nut oil, mango butter, marula oil, meadowfoam oil, mid chain tryglycerides liquid (mct), mineral oil, moringa seed oil, murumuru oil, Oenothera biennis oil, olive oil, orange oil, Orblignya oleifera seed oil, palm oil, pamergranet seed oil, papaya seed oil, paraffinum liquidum Persea gratissima oil, peach kernel oil, perilla oil, petrolatum, polyglycerin-3, primerose oil, pumpkin seed oil, Punica granatum seed oil, rasberry seed oil, rice bran oil, rosehip oil, safflower oil, sal butter, Schinziophyton rautanenil seed oil, sesame oil, shae butter oil, shorea (sal) butter oil, sorbitol, soybean oil, sunflower oil, squalene oil, tamanu oil, terpene oil, Theobroma grandiflorum seed butter, tomato seed oil, vegetable oil, walnut oil, and wheat germ ol.

8. The lip scrub composition of claim 6, wherein the lip scrub composition comprises about 1% to about 3% w/w of the flavoring agent(s), and wherein the flavoring agent is selected from: sugar-free powder flavor, sugar-free flavor oil, sugar-free extract flavor, sugar-free sweetener, green tea powder, and fine coffee ground.

9. The lip scrub composition of claim 6, wherein the lip scrub composition comprises about 0% to about 15% w/w of the thickening agent, and wherein the thickening agent is selected from almond butter, aloe butter, argon butter, avocado butter, beeswax, candelilla wax, carnauba wax, chamomile, cocoa butter, coconut butter, coffee butter, carrot seed butter, cranberry butter, cupuacu butter, emulsifying wax, Euphorhia cerifera wax, grean tea butter, hemp butter, hempseed butter, horsetail butter, jojoba butter, jojoba wax, kokum butter, lanolin butter, lavender butter, lemon butter, macadamia butter, mango butter, olive butter, orange butter, ozokerite wax, palm butter, polysorbate, pumpkin seed butter, sal butter, shae butter, shorea (sal) butter, soy butter, Theobroma grandifiorum (Cupuacu) seed butter, tomato seed butter, tucuma butter, and tumeric butter.

10. The lip scrub composition of claim 6, wherein the lip scrub composition comprises about 0.5% to about 1% w/w of the essential oil, and wherein the essential oil is selected from citral, grapefruit, lavender, lemon, lime, orange, peppermint, terpene oil, vanilla, and wintergreen.

11. The lip scrub composition of claim 6, wherein the lip scrub composition comprises about 0% to about 1% w/w of the colorant, and wherein the colorant is selected from food grade colorants powder or oil, sugar free colorants powder or oil, micas, pigment, starch, and liquid dye.

12. The lip scrub composition of claim 6, wherein the lip scrub composition comprises about 0% to about 1% w/w of the vitamin, and wherein the vitamin is selected from vitamin C, vitamin E, and vitamin K1.

13. The lip scrub composition of claim 6, wherein the lip scrub composition comprises about 0% to about 10% w/w of the humectant, and wherein the humectant is selected from: Crambe abyssinica seed oil, glycerin, lecithin, and propylene glycol.

14. The lip scrub composition of claim 6, wherein the lip scrub composition comprises about 0.15% to 0.3% w/w of the preservative, and wherein the preservative is selected from: benyzl alcohol, dehydroacetic acid, glyceryl caprilate, hydroxyacetophenone, potassium sorbite, caprylhydroxamic acid, caprylyl glycol, lycerin, geogard, ECT, sensicare M4200, Geogard Ultra, anisic acid, glyceryl caprylate, glyceryl undecylenate, naticide, ehtyl lauroyl arginate, and biosecur.

15. The lip scrub composition of claim 1, further comprising an abrasive that is not a sugar alcohol or a sugar.

16. The lip scrub composition of claim 15, wherein the lip scrub composition comprises about 0% to about 15% w/w of the abrasive, and wherein the abrasive is selected from sugar-free sprinkles nonpareil, fruit peel, fruit powder, vegetable peel, vegetable powder, and jojoba beads.

17. The lip scrub composition of claim 1, further comprising:
(i) a lip plumping agent selected from sodium hyaluronate, cinnamon, capsicum, and menthol; or
(ii) cannabidol (CBD) oil.

18. The lip scrub composition of claim 1, wherein the lip scrub composition is formulated as a lip balm, lip stick, lip cream, lip gel, or lip gloss.

19. A method of exfoliating lips of a subject, comprising topically administering to the lips of the subject the lip scrub composition of claim 1.

20. The method of claim 19, wherein the lip scrub composition is applied directly to the lips, applied to the lips via an applicator, or rubbed onto the subject's fingers and then applied to the lips.
